(19) United States
(12) Patent Application Publication
(10) Pub. No.: US 2007/0104774 A1
(43) Pub. Date: May 10, 2007

(54) Method for preparing phytosphingosine liposome composition

(76) Inventors: Sunki Kim, Seoul (KR); Sangwoo Cho, Seoul (KR); Eunok Lee, Seoul (KR); Eunhee Yang, Seoul (KR); Myungjun Choi, Seoul (KR)

Correspondence Address: DICKSTEIN SHAPIRO LLP, 1825 EYE STREET NW, Washington, DC, 20006-5403, US

(21) Appl. No.: 10/579,081

(22) PCT Filed: Nov. 11, 2004
(86) PCT No.: PCT/KR04/02920

§ 371 Date: May 11, 2006

(30) Foreign Application Priority Data
Nov. 11, 2003 (KR) 10-2003-0079419
Nov. 3, 2004 (KR) 10-2004-0088891

(51) Int. Cl. A61K 9/127

(52) U.S. Cl. 424/450

(57) ABSTRACT

The invention relates to a method for preparing phytosphingosine liposome composition having excellent long-term storage stability and the method comprises dispersing phytosphingosine in water, separately dissolving phospholipid in a solvent and mixing the two solutions prepared by the above procedures.

### TECHNICAL FIELD

The present invention relates to a method for preparing a water-soluble composition containing phytosphingosine, which is a water-insoluble active ingredient, more particularly, to a composition containing phytosphingosine having a dispersion-stability by liposome.

### BACKGROUND ART

Phytosphingosine is a kind of sphingolipids, one of main ingredients constituting a cell membrane of a living body, and a physiologically active substance having various biological functions as a signal transmitter of a signal transduction system as well as structural functions (Okazaki et al., 1989; Kim et al., 1991). Phytosphingosine and ceramide types having a backbone of the phytosphingosine have been used as functional raw materials for a skin moisturizing function and a regeneration of damaged skin-protecting film in cosmetics industries. It is expected that phytosphingosine will be more used since it has excellent skin generation, antibacterial and anti-inflammatory effects, compared to ceramide. Recently, phytosphingosine is more easily obtained by yeast fermentation, instead of being extracted from an animal. However, since phytosphingosine obtained by yeast fermentation has also a poor solubility, it has a limitation when applying it as cosmetic and pharmaceutical uses. Further, since phytosphingosine is never dissolved in water and dissolved in an isocetyl alcohol solvent only by 1 wt. %, which is usable as raw materials for cosmetics, it is difficult to use phytosphingisine as functional raw materials for cosmetics in an amount enough to exhibit effects and it is impossible to use phytosphingosine in a transparent solution product such as water lotions. Since a concentration of phytosphingosine currently used in cosmetics is 0.1˜0.3 wt. % which is much lower than a concentration of 0.5 wt. % required for exhibiting a sufficient effect, it is difficult to sufficiently obtain a desired effect. Accordingly, in order to obtain the optimum effect, it is required to develop a method of dissolving phytosphingosine in a high concentration and stabilizing it for a long time.

In order to dissolve materials having a low solubility such as phytosphingosine, a method such as micelle, nano-capsule, emulsion and liposome, etc. is used. The method varies with the uses or usable materials, etc. Korean Patent Unexamined Publication Numbers 2002-32577 titled “Compositions and Methods for Controlling Content of Lipid of Skin” and 2000-75480 titled “Aqueous Pharmaceutical Composition containing an Active Ingredient which is highly insoluble in water” disclose that liposome is used for dissolving an active material having a low solubility. Liposome has advantages in that phospholipid used for preparing liposome is a natural material and forms a lamellar structure and thus has an effect of skin moisturizing. Liposome is a globular water-soluble particle surrounded by one or more lipid bilayer and has various sizes of several tens nm˜several microns, so that it can be diversely used according to its purposes. Liposome can collect both water-soluble and fat-soluble materials, be easily prepared and transfer an active material, so that it is broadly applied in preparing a drug or cosmetics and researched for dissolving and delivering poorly soluble active materials. It is required to stabilize an active material having a low solubility by applying liposome to cosmetics. In addition, it is possible to easily penetrate into skin by using small-sized liposome, so that liposome including a high concentration of phytosphingosine and having a small size of 100 nm or less is prepared and can be used to water-soluble cosmetics restrained from using phytosphingosine and easily applied to existing cream products. Thus, its effects can be maximized due to the ability of penetrating into the skin in addtion to convenience in use.

Korean Patent Registration No. 10-343885 (Method for Preparing Aqueous Solution of Phytosphingosine) discloses that phytosphingosine is dissolved in water in a high concentration. However, when phytosphingosine is dissolved with lactic acid and a liquid extract extracted from willow bark, there is a problem of an occurrence of sedimentation when it is stored for a long time.

### DISCLOSURE OF INVENTION

Accordingly, the present invention has been made to solve the above-mentioned problems occurring in the prior art. The object of the present invention is to make it possible for phytosphingosine to be contained in a high concentration in transparent solution products such as water lotions by stably dissolving phytosphingosine having a low solubility in water in a high concentration for a long time.

In order to accomplish the objects, the method for preparing phytosphingosine liposome composition comprises steps of (1) dispersing phytosphingosine in water and adding lactic acid to the dispersed solution to dissolve phytosphingosine; (2) dissolving phospholipid in a solvent; (3) mixing the solution prepared from the step (1) and the solution prepared from the step (2); (4) emulsifying the mixture obtained from the step (3); and (5) extruding the mixture emulsified in the step (4).

According to the invention, a content of phytosphingosine of the step (1) is preferably 0.1˜10 wt. % of the total liposome composition.

According to the invention, phospholipid of the step (2) is at least one selected from a group consisting of natural or hydrogenated phosphatidylcholine, phosphatidylethanolamine, phosphatidylinositol, phosphatidylserine, shpingomyeline, lyso-phosphatidylcholine and lyso-phosphatidylethanolamine derived from soybean or yolk phospholipid.

According to the invention, a content of phospholipid of the step (2) is preferably 2˜20 wt. % of the total liposome composition.

According to the invention, the solvent of the step (2) is preferably selected from a group consisting of low alcohol, diol and a mixture thereof.

According to the invention, the solvent of the step (2) is preferably 1˜50 wt. % of the total liposome composition.

According to the invention, the emulsified mixture is extruded preferably through a membrane having pores of 200 nm or less in the step (5).

According to another embodiment of the invention, the cosmetic composition contains the phytosphingosine liposome composition prepared by the above methods.

According to the invention, the cosmetic composition contains 0.1˜20wt. % of the phytosphingosine liposome composition of the total cosmetic composition.

### BRIEF DESCRIPTION OF THE DRAWINGS

The above and other objects, features and advantages of the present invention will be more apparent from the following detailed description taken in conjunction with the accompanying drawings, in which:

FIG. 1 shows an observatory result of phytosphingosine liposome compositions prepared according to an embodiment of the invention; and

FIG. 2 shows an observatory result of cosmetic compositions prepared according to embodiments of the invention.

### DETAILED DESCRIPTION OF THE PREFERRED EMBODIMENTS

Hereinafter, preferred embodiments of the present invention will be described with reference to the accompanying drawings. In the following description of the present invention, a detailed description of known functions and configurations incorporated herein will be omitted when it may make the subject matter of the present invention rather unclear.

According to the invention, the method of preparing transparent and stable liposome containing phytosphingosine comprises dissolving phytosphingosine in an aqueous solution using lactic acid, dissolving phospholipid in a solvent, and mixing the solutions of the two phases, emulsifying and extruding the mixture prepared.

More specifically, phytosphingosine is added to distilled water in a concentration of 0.1˜10 wt. %, more preferably 0.5˜5wt. % of a total liposome composition, warmed to a temperature of 70° C. and then stirred for 30˜40 minutes. Here, when the content of phytosphigosine is less than 0.1 wt. % of the total liposome composition, it is difficult to expect an effect as an effective ingredient, and when the content is more than 10 wt. %, phytosphingosine is precipitated. Accordingly, it is preferred that the content of phytosphingosine is 0.5˜10 wt. %. In this regard, the content of phytosphingosine of 0.5˜5 wt. % is most preferable. When phytosphingosine is dispersed in water as mentioned above, pH thereof becomes basic. In order to dissolve phytosphingosine by lowering pH, pH of the solution is neutralized by slowly adding lactic acid, and the solution is stirred until phytosphingosine is completely dissolved. It is preferred that lactic acid is added in an amount of 0.2˜4 g per 1 g of phytosphingosine. When the amount of lactic acid is less than 0.2 g, phytosphingosine is not dissolved, and when the amount is more than 4 g, pH of the solution is too lowered.

Then, phospholipid is dissolved in a solvent in a concentration of 2˜20 wt. % of the total liposome composition, separately from the above-mentioned aqueous solution preparation. When a content of phospholipid is less than 2 wt. %, phytosphingosine is not stabilized, and when the content is more than 20 wt. %, a viscosity of the solution is too high, so that it is difficult to use it. The solution prepared like this is mixed with the phytosphingosine aqueous solution and emulsified. A typical method used for conventional liposome preparations can be used for this emulsification. Here, refined soybean phospholipid and yolk phospholipid are used as phospholipid. Soybean phospholipid and yolk phospholipid contain phosphatidylcholine, phosphatidylethanolamine, phosphatidylinositol, phosphatidylserine, sphingomyeline, lyso-phosphatidylcholine and lyso-phosphatidylethanolamine, etc. It is preferred to add the solvent in an amount of 1˜50 wt. % of the total liposome composition. The emulsified liposome solution is extruded to 0.2 μm or less two times or more, so that the size of liposome is maintained to be 100 nm or less and thus it is possible to easily penetrate into the skin. The extrusion is a process of passing the solution through a membrane having pores of a predetermined size under pressure.

In the liposome composition of the invention, it is preferred that a content of the solvent is 1˜50 wt. % of the total liposome composition. It is possible to easily dissolve phospholipid when the content of the solvent is more than 1 wt. %, and the liposome composition gelled when the content of the solvent is more than 50 wt. %.

In the liposome composition of the invention, low alcohol used as the solvent can be, for example, ethanol, propanol, buthanol, etc. Preferred low alcohol is ethyl alcohol.

In the liposome composition of the invention, diol used as the solvent can be, for example, ethylene glycol, propylene glycol, butylene glycol, etc. Preferred diol may be 1,3-buiylene glycol and propylene glycol, etc.

In the specification, “wt. %” used for contents of ingredients regarding the explanation of the liposome composition means a weight percent of the total water solution, and “wt. %” used for contents in cosmetics means a weight percent of the total cosmetic composition.

The liposome composition of the invention is characterized in that phytosphingosine is dissolved in water and phospholipid is separately dissolved in a solvent and then the two solutions are mixed. Typically, a fat-soluble material such as phytosphingosine has been dissolved in a solvent. However, when phytosphingosine is dissolved in the solvent, it exhibits a viscosity in water-soluble phase due to its peculiar properties, so that it is difficult to use it due to its high viscosity. In addition, when phytosphingosine is directly dissolved in the solvent and thus stabilized, a phase of other forms, rather than a liposome form, is made, so that the stabilization using liposome becomes impossible. The inventors found these problems, and adopted a new method of dissolving phytosphingosine in water, separately dissolving phospholipid in a solvent and then mixing the two solutions, in order to solve the problems.

Hereinafter, the invention will be more specifically explained by describing preferred embodiments of the present invention. However, the following embodiments are provided only to more fully disclose the invention and not intended to limit the scope of the invention.

### EXAMPLE

### Example 1

1.35 g of phytosphingosine powder (available from Doosan Corporation, trade name DS-phytosphingosine) were added to 93 ml of distilled water, and stirred at 100 rpm while slowly warming to 70° C. Phytosphingosine was completely dissolved while neutralizing pH of the solution by adding 0.4 g of lactic acid. Separately, 3 g of refined soybean phospholipid was dissolved in 2 g of ethyl alcohol. The prepared phospholipid solution was mixed with the distilled water in which phytosphingosine was dissolved. Then, the mixture was subject to an ultrasonic treatment. The ultrasonic-treated liposome solution was extruded to 0.1˜0.2 μm two times, so that a transparentr phytosphingosine liposome solution having an average particle size of 0.1 μm of liposome was obtained.

The refined soybean phospholipid used in the above example had the following composition.

TABLE 1

| Ingredients | wt. % |
|---|---|
| Phosphatidylcholine | 70˜95 |
| Phosphatidylethanolamine | 0˜10 |
| Phosphatidylinositol | 0˜2 |
| Lyso-phosphatidylcholine | 0˜5 |

### Example 2

1.35 g of phytosphingosine (available from Doosan Corporation, trade name DS-phytosphingosine) was added to 93 ml of distilled water, and stirred at 100 μm while slowly warming to 70° C. Phytosphingosine was completely dissolved while neutralizing the pH of the solution by adding 0.4 g of lactic acid. Separately, 3 g of refined yolk phospholipid was dissolved in 2 g of ethyl alcohol. The prepared phospholipid solution was mixed with the distilled water in which phytosphingosine was dissolved. Then, the mixture was subject to an ultrasonic treatment. The ultrasonic-treated liposome solution was extruded to 0.1˜0.2 μm two times, so that a transparent phytosphingosine liposome solution having an average particle size of 0.1 μm of liposome was obtained.

The refined yolk phospholipid used in the above example had the following composition.

TABLE 2

| Ingredients | wt. % |
|---|---|
| Phosphatidylcholine | 70˜95 |
| Phosphatidylethanolamine | 0˜20 |
| Lyso-phosphatidylcholine | 0˜3 |
| Lyso-phosphatidylethanolamine | 0˜5 |
| Sphingomyeline | 0˜5 |

### Example 3

4 g of phytosphingosine (available from Doosan Corporation, trade name DS-phytosphingosine) was added to 73 ml of distilled water, and stirred at 100 rpm while slowly warming to 70° C. Phytosphingosine was completely dissolved while neutralizing the pH of the solution by adding 1 g of lactic acid. Separately, 12 g of refined soybean phospholipid was dissolved in 10 g of ethyl alcohol. The prepared phospholipid solution was mixed with the distilled water in which phytosphingosine was dissolved. Then, the mixture was emulsified three times under a 300 bar of pressure. The high-pressure emulsified liposome solution was extruded to 0.1˜0.2μm two times, so that a transparent phytosphingosine liposome solution having an average particle size of 70 nm of liposome was obtained.

### Example 4

4 g of phytosphingosine (available from Doosan Corporation, trade name DS-phytosphingosine product) was added to 73 ml of distilled water, and stirred at 100 rpm while slowly warming to 70° C. Phytosphingosine was completely dissolved while neutralizing the pH of the solution by adding 1 g of lactic acid. Separately, 12 g of hydrogenated, refined soybean phospholipid, in which the unsaturated fatty acid was transformed to oleic acid by a partial hydrogenation, was dissolved in 10 g of ethyl alcohol. The prepared phospholipid solution was mixed with the distilled water in which phytosphingosine was dissolved. Then, the mixture was subject to an ultrasonic treatment. The ultrasonic-treated liposome solution was extruded to 0.1˜0.2 μm two times, so that a transparent phytosphingosine liposome solution having an average particle size of 50 nm of liposome was obtained. The content of oleic acid of the partially hydrogenated, refined soybean phospholipid used was 50% or more.

### Example 5

4 g of phytosphingosine (available from Doosan Corporation, trade name DS-phytosphingosine) was added to 73 ml of distilled water, and stirred at 100 rpm while slowly warming to 70° C. Phytosphingosine was completely dissolved while neutralizing the pH of the solution by adding 1 g of lactic acid. Separately, 12 g of hydrogenated, refined soybean phospholipid was dissolved in 10 g of ethyl alcohol. The prepared phospholipid solution was mixed with the distilled water in which phytosphingosine was dissolved. Then, the mixture was subject to an ultrasonic treatment. The ultrasonic-treated liposome solution was extruded to 0.1˜0.2 μm two times, so that a semitransparent phytosphingosine liposome solution having an average particle size of 100 nm of liposome was obtained. Phospholipid, in which unsaturated fatty acid was little and saturated fatty acid was major, was used as the hydrogenated, refined soybean phospholipid.

### Example 6

4 g of phytosphingosine was added to 73 ml of distilled water, and stirred at 100 rpm while slowly warming to 70° C. Phytosphingosine was completely dissolved while neutralizing the pH of the solution by adding 1 g of lactic acid. Separately, 12 g of refined soybean phospholipid was dissolved in 10 g of 1,3-butylene glycol. The prepared phospholipid solution was mixed with the distilled water in which phytosphingosine was dissolved. Then, the mixture was emulsified three times under a 300 bar of pressure. The high-pressure emulsified liposome solution was extruded to 0.1˜0.2 μm two times, so that a transparent phytosphingosine liposome solution having an average particle size of 60 nm of liposome was obtained.

### Example 7

4 g of phytosphingosine was added to 53 ml of distilled water, and stirred at 100 rpm while slowly warming to 70° C. Phytosphingosine was completely dissolved by adding 2 g of lactic acid. Separately, 12 g of refined soybean phospholipid was dissolved in 30 g of 1,3-butylene glycol. The prepared phospholipid solution was mixed with the distilled water in which phytosphingosine was dissolved. Then, the mixture was subject to an ultrasonic-treatment. The ultrasonic-treated liposome solution was extruded to 0.1˜0.2 μm two times, so that a transparent phytosphingosine liposome solution having an average particle size of 50 nm of liposome was obtained.

### Example 8

4 g of phytosphingosine was added to 58 ml of distilled water, and stirred at 100 rpm while slowly warming to 70° C. Phytosphingosine was completely dissolved by adding 1 g of lactic acid. Separately, 12 g of refined soybean phospholipid was dissolved in 25 g of propylene glycol. The prepared phospholipid solution was mixed with the distilled water in which phytosphingosine was dissolved. Then, the mixture was subject to an ultrasonic-treatment. The ultrasonic-treated liposome solution was extruded to 0.1˜0.2 μm two times, so that a transparent phytosphingosine liposome solution having an average particle size of 50 nm of liposome was obtained.

### Comparative example 1

1.35 g of phytosphingosine powder (available from Doosan Corporation, trade name DS-phytosphingosine) and 3 g of refined soybean phospholipid were dissolved in 3.5 g of ethyl alcohol. To the prepared solution was added 93 ml of distilled water, and then the mixture was subject to an ultrasonic treatment. The inventors tried to extrude the ultrasonic-treated liposome solution to 0.1˜0.2 μm two times. However, the solution prepared according to the above procedure could not be extruded.

### EXPERIMENTAL EXAMPLE 1

### Observation of a Suspended Degree of Phytosphingosine Liposome Composition

The inventors performed an observation of a suspended degree so as to examine transparencies of the liposome composition of the invention and the cosmetic composition containing the liposome composition.

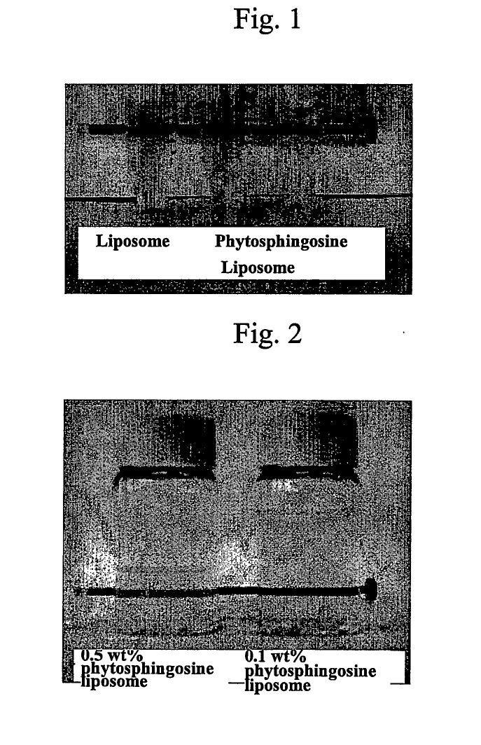

FIG. 1 shows an observatory result of a phytosphingosine liposome composition prepared according to the Example 3. As shown in FIG. 1, it can be seen that it was obtained a transparent solution exhibiting an inherent color of phospholipid.

FIG. 2 shows an observatory result of a cosmetic composition according to formulation 2 of a following experimental example 3. As shown in FIG. 2, when a cosmetic composition was prepared by diluting the liposome composition, it was obtained a semitransparent solution exhibiting a blue color.

### EXPERIMENTAL EXAMPLE 2

### a Stability Test of Phytosphingosine Liposome

The liposome solutions prepared according to the Examples 1 to 8 were examined in views of variations of particle sizes and a sedimentation formation as time went by. In order to carry out this examination, the liposome solutions having a high concentration of phytosphingosine and liposome solutions diluted in distilled water about by ten times, respectively were prepared. The respective liposome solutions were stored at room temperature (R.T.) and it was observed variations of particle sizes and a sedimentation formation as time went by. The results were shown in Table 3. In the Table, a unit of a particle size is nm, ‘1’ denotes a high concentration of liposome solution state and ‘2’ denotes a diluted state.

TABLE 3

| Storage period of | Example 1 | | Example 2 | | Example 3 | | Example 4 | | Example 5 | | Example 6 | | Example 7 | | Example 8 | |
|---|---|---|---|---|---|---|---|---|---|---|---|---|---|---|---|---|
| sample (day) | 1 | 2 | 1 | 2 | 1 | 2 | 1 | 2 | 1 | 2 | 1 | 2 | 1 | 2 | 1 | 2 |
| 0 | 92 | 100 | 87 | 117 | 42 | 84 | 53 | 99 | 94 | 101 | 66 | 78 | 44 | 65 | 57 | 78 |
| 7 | 96 | 105 | 85 | 119 | 39 | 84 | 51 | 86 | 93 | 110 | 61 | 86 | 47 | 68 | 58 | 82 |
| 21 | 99 | 108 | 94 | 120 | 45 | 79 | 51 | 86 | 92 | 111 | 59 | 79 | 48 | 68 | 54 | 85 |
| 60 | 104 | 111 | 95 | 126 | 52 | | 52 | | | | 60 | 82 | 53 | 67 | 57 | 82 |
| 90 | 100 | 109 | 98 | 128 | 56 | 88 | 51 | 88 | | | 62 | 85 | 47 | 77 | 60 | 85 |

As shown in the Table, the phytosphingosine liposome solution prepared according to the invention maintained its stable state without a large variation of liposome sizes and a sedimentation formation as time went by, under a state that 1.35 wt. % and 4 wt. % of phytosphingosine was contained in the liposome. Contrary to this, when phytosphingosine was directly dissolved in a solvent (Comparative example 1), it was impossible to carry out a process of extrusion for the sample 1 and thus it was improper for a practical use.

### EXPERIMENTAL EXAMPLE 3

### a Stability Test of Water Lotion Formulation of Phytosphingosine Liposome

For a liposome solution containing 4 wt. % of phytosphingosine prepared according to Examples 3 and 7 and a solution in which the liposome solution was diluted so as to make a final concentration of phytosphingosine be 0.5% in a water lotion product, it was examined a stability of phytosphingosine liposome in water lotion formulation, based on sizes and sedimentation states as time went by and sizes and sedimentation states when storing at R.T. and a low temperature, respectively.

Water lotion formulations used in the above test are as follows. The unit is wt. %.

TABLE 4

| | |
|---|---|
| Formulation 1 | Formulation 2 |

Water-phase part

| | | |
|---|---|---|
| Glycerin | 4 | 4 |
| 1,3-butyleneglycol | 2 | 2 |
| Allantoin | 0.1 | 0.1 |
| DL-panthenol | 0.1 | 0.1 |
| EDTA | 0.01 | 0.01 |
| Bio-he | | 0.5 |
| Camomile | | 0.5 |
| Bg 100 | | 0.1 |

Alcohol part

| | | |
|---|---|---|
| Ethyl alcohol | 6 | 6 |
| D-M | 0.15 | 0.15 |
| Surfactant | 0.2 | 0.2 |
| Incense | qs | 0.1 |
| Purified water | to 100 | to 100 |

In water lotion formulations of liposome containing phytosphingosine, results of size variations as time went by are as follows. The unit is nm.

TABLE 5

| | | | | | 0.5% phytosphingosine liposome | | | | 0.5% phytosphingosine liposome | | | |
|---|---|---|---|---|---|---|---|---|---|---|---|---|
| | 4% phytosphingosine liposome | | | | formulation 1 | | | | formulation 2 | | | |
| | Example 3 | | Example 7 | | Example 3 | | Example 7 | | Example 3 | | Example 7 | |
| Storage period | (ethanol) | | (butylene glycol) | | (ethanol) | | (butylene glycol) | | (ethanol) | | (butylene glycol) | |
| of sample (day) | 4 | R.T. | 4 | R.T. | 4 | R.T. | 4 | R.T. | 4 | R.T. | 4 | R.T. |
| 0 | 76 | 76 | 44 | 44 | 82 | 82 | 46 | 46 | 88 | 88 | 52 | 52 |
| 7 | 75 | 75 | 48 | 47 | 73 | 77 | 51 | 52 | 76 | 78 | 51 | 53 |
| 14 | 75 | 75 | 46 | 48 | 75 | 78 | 49 | 50 | 75 | 79 | 51 | 53 |
| 50 | 76 | 74 | 46 | 53 | 71 | 79 | 49 | 53 | 73 | 78 | 52 | 53 |
| 100 | 75 | 75 | 45 | 47 | 76 | 74 | 52 | 57 | 79 | 79 | 51 | 54 |

As shown in the Table, in the case of liposome containing 4 wt. % of phytosphingosine, the stability was maintained without size variations and sedimentation. Further, in the case of the water lotion liposome compsositions (Formulation 1 and 2) containing phytosphingosine at a concentration of 0.5 wt. %, at which phytosphingosine effect can be obtained, their stability were maintained without size variations and sedimentation, in both water lotion formulations classified whether plant extracts were contained or not.

When phytosphingosine was dissolved using an acid only such as lactic acid, it was recrystallized as time went by and in storing at low temperatures. However, when phytosphingosine was contained in liposome and thus stabilized as disclosed in the invention, it was maintained as a transparent solution or a transparent gel state in spite of storage at low temperatures or even though time passed.

In addition, phytosphingosine stabilized with liposome was not precipitated when storing at low temperatures or for a long time and exhibited a stability maintaining its initial size. Further, even when a high concentration of phytosphingosine was diluted, it maintained a stable liposome and exhibited a stability maintaining a transparent state without sedimentation.

As described above, the phytosphingosine liposome composition prepared according to the invention provides convenience in its use since it can be used in cosmetics at high concentrations while maintaining inherent functions of phytosphingosine such as antibacterial, anti-inflammatory and skin generation effects. In addition, according to the invention, it is possible to provide a liposome solution having a stability exhibiting no sedimentations of phytosphingosine, which can occur when it is stored for a long time or at low temperatures. Additionally, since phospholipid used in the invention forms a lamellar structure and thus has an effect of skin moisturizing, the moisturizing function is improved as well as the inherenct functions of phytosphingosine when the liposome composition prepared according to the invention is used in cosmetic composition.

Further, since the liposome composition forms a transparent or translucent solution when it is diluted in water lotion formulations, it can be easily applied to water-soluble cosmetic formulations such as water lotion formulations and essences etc, in which phytosphingosine has not been used due to its poor solubility, as well as existing cream products which has used phytosphingosine powders. Since the phytosphingosine liposome prepared according to the invention has a size of 100 nm or less, when it is applied to the skin, it can easily penetrate into the skin, rather than simply being applied to the epidermis. Accordingly, it is possible to add a transporter role of phytosphingosine to the liposome.

While the invention has been shown and described with reference to certain preferred embodiments thereof, it will be understood by those skilled in the art that various changes in form and details may be made therein without departing from the spirit and scope of the invention as defined by the appended claims.

What is claimed is:

1. A method for preparing phytosphingosine liposome composition comprising:
(1) dispersing phytosphingosine in water and adding lactic acid to the dispersed solution to dissolve phytosphingosine;
(2) dissolving phospholipid in a solvent;
(3) mixing the solution prepared from the (1) and the solution prepared from the (2);
(4) ultrasonic-treating or emulsifying the mixture obtained from the (3); and
(5) extruding the mixture treated or emulsified in the (4).

2. The method according to claim 1, wherein a content of phytosphingosine of the (1) is 0.1˜10 wt. % of the total liposome composition.

3. The method according to claim 1, wherein the phospholipid of the (2) is at least one selected from the group consisting of natural or hydrogenated phosphatidylcholine, phosphatidylethanolamine, phosphatidylinositol, phosphatidylserine, shpingomyeline, lyso-phosphatidylcholine and lyso-phosphatidylethanolamine derived from soybean or yolk phospholipid.

4. The method according to claim 1, wherein a content of phospholipid of the (2) is 2˜20wt. % of the total liposome composition.

5. The method according to claim 1, wherein the solvent of the (2) is selected from the group consisting of low alcohol, diol and a mixture thereof.

6. The method according to claim 5, wherein a content of the solvent is 1˜50wt. % of the total liposome composition.

7. The method according to claim 1, wherein the mixture is extruded through a membrane having pores of 200 nm or less in the (5).

8. A cosmetic composition containing the phytosphingosine liposome composition prepared according to any one of claims 1 to 7.

9. The composition according to claim 8, wherein the cosmetic composition contains 0.1˜20 wt. % of the phytosphingosine liposome composition of the total cosmetic composition.
